(19) United States
(12) Patent Application Publication
(10) Pub. No.: US 2009/0306647 A1
(43) Pub. Date: Dec. 10, 2009

(54) DYNAMICALLY CONTROLLABLE MULTI-ELECTRODE APPARATUS & METHODS

(76) Inventors: Greg Leyh, Brisbane, CA (US); Jerzy Orkiszewski, Palo Alto, CA (US); Richard Canant, Pacifica, CA (US)

Correspondence Address: Cutera c/o MoFo, 425 Market Street, San Francisco, CA, 94105-2482, US

(21) Appl. No.: 12/134,119

(22) Filed: Jun. 5, 2008

(51) Int. Cl. A61N 1/08

(52) U.S. Cl. 606/34

(57) ABSTRACT

Apparatus and methods for dynamically controlling a plurality of electrodes during an electrosurgical procedure, wherein each electrode may be controlled with respect to active or return electrode mode, condition, and power level. The electrodes may be disposed within a treatment chamber of a handpiece. Each electrode may comprise a spiral inductor. The handpiece may be equipped with suction and vibration means. The treatment chamber may be configured for receiving at least a portion of the target tissue therein.

### FIELD OF THE INVENTION

The present invention generally relates to apparatus and methods for electrosurgery.

### BACKGROUND OF THE INVENTION

Cellulite is a common skin condition related to the accumulation of excess subcutaneous fat (adipose tissue) within fibrous septae. Irregularities in the structure of the fibrous septae can create the appearance of cellulite, which is typically seen as an unsightly irregular, dimpled skin surface. Cellulite is often found in abundance in overweight and obese individuals, e.g., on the thighs, hips and buttocks. The proportion of children, adolescents, and adults who are overweight or obese is increasing. The number of overweight people has doubled in the last two to three decades, and such increases are found in all age, race, and gender groups. As a result, there is a large demand for treatments that will decrease the appearance of cellulite for cosmetic purposes. There is also a demand for apparatus and procedures that will reduce the overall volume of adipose tissue and/or reshape subcutaneous fat.

Prior art interventions for decreasing or reshaping adipose tissue include liposuction and lipoplasty, massage, low level laser therapy, and external topical compositions, such as “cosmeceuticals,” or a combination of such treatments. Liposuction and lipoplasty are invasive surgical techniques in which subcutaneous fat is excised and/or suctioned from the body. These procedures may be supplemented by the application to the targeted adipose tissue of various forms of energy to emulsify the fat prior to its removal, e.g., by suction.

Although liposuction and lipoplasty can effectively remove subcutaneous fat, the invasive nature of these procedures presents the inherent disadvantages of surgery, including high cost and extended recovery times, as well as associated risks such as infection, excessive bleeding, and trauma.

Non-invasive interventions for subcutaneous fat reduction, or diminution of the appearance of cellulite, including massage and low-level laser therapy, are significantly less effective than surgical intervention.

Some cosmetic skin treatments effect localized dermal heating by applying radiofrequency (RF) energy to the skin using surface electrodes. The local heating is intended to tighten the skin by producing thermal injury that changes the ultrastructure of collagen in the dermis, and/or results in a biological response that changes the dermal mechanical properties. The literature has reported some atrophy of sub-dermal fat layers as a complication to skin tightening procedures.

US Patent Application Publication No. 20060036300 (Kreindel) discloses lipolysis apparatus having one or more terminal electrodes protruding from an RF applicator. In lipolysis methods of Kreindel, a region of tissue may be deformed, and the electrodes may contact both deformed and non-deformed skin. In an embodiment of Kreindel, light energy (e.g., from a laser) is applied so as to penetrate beneath the dermal layer.

It can be seen that there is an ongoing need for an effective modality by which subcutaneous fat tissue may be non-invasively reshaped, and/or sculpted for the cosmetic improvement of human skin and/or body shape. There is a further need for a non-invasive procedure for effectively and efficiently decreasing the volume of subcutaneous adipose tissue in a person who may be obese or overweight.

### SUMMARY OF THE INVENTION

According to one aspect of the invention, there is provided a system for treating a patient, wherein the system comprises a handpiece configured for contacting a target region of skin of the patient, and an electrosurgical generator coupled to the handpiece. The handpiece includes a treatment surface and a plurality of electrodes disposed on the treatment surface. The system is configured for independently dynamically controlling at least one of: mode of operation, power level, and condition for each of the electrodes.

According to another aspect of the invention, there is provided apparatus for treating a patient, wherein the apparatus comprises a handpiece including a plurality of electrodes. Each of the electrodes comprises a spiral inductor, and at least two of the spiral inductors are disposed in at least two different planes.

According to a further aspect of the invention, apparatus for treating a patient comprises a handpiece having a treatment surface, and a plurality of electrodes disposed on the treatment surface. The handpiece is configured for contacting the treatment surface against the skin of the patient, and each of the electrodes is configured for independent dynamic control with respect to at least one of mode of operation, power level, and condition.

According to still another aspect of the invention, there is provided a handpiece for treating a target tissue of a patient. The handpiece comprises a shell having a treatment chamber therein, a treatment surface within the treatment chamber, and a plurality of electrodes disposed on the treatment surface. The treatment chamber is configured for receiving the target tissue, the treatment surface is configured for contacting the skin of the patient against the electrodes, and each of the electrodes is configured for independent dynamic control with respect to at least one of mode of operation, power level, and condition.

According to still a further aspect of the invention, there is provided a method for treating a patient, wherein the method comprises providing a handpiece having a plurality of electrodes; applying electrical energy to a target tissue of the patient via at least one of the electrodes; and during the applying step, independently dynamically controlling each of the electrodes with respect to at least one of mode of operation, power level, and condition. At least one of the electrodes comprises a spiral inductor, and the spiral inductor is at least substantially planar.

According to yet another aspect of the invention, a method for selectively heating a target tissue of a patient comprises providing a handpiece having a plurality of electrodes, a treatment chamber, and a flange; contacting the flange against the patient's skin, such that the flange surrounds a target region of the patient's skin; drawing the target tissue into the treatment chamber; and during the drawing step, applying electrical energy to the target tissue via the electrodes. The electrodes are disposed on a treatment surface within the treatment chamber, each of the electrodes comprises a spiral inductor, and each the spiral inductor is substantially planar. The target tissue may comprise subcutaneous fat disposed beneath the target region of the patient's skin.

These and other features, aspects, and advantages of the present invention may be further understood with reference to the drawings, description, and claims which follow.

### BRIEF DESCRIPTION OF THE DRAWINGS

FIG. 1A schematically represents an electrosurgical procedure for treating a patient using a handpiece having a plurality of electrodes, according to an embodiment of the invention;

FIG. 1B schematically represents an electrosurgical procedure for treating a patient using a handpiece having a plurality of electrodes, according to another embodiment of the invention;

FIG. 2A is a block diagram schematically representing an electrosurgical system having a plurality of independently controllable electrodes, according to an embodiment of the invention;

FIG. 2B is a block diagram schematically representing an electrosurgical system including a plurality of independently controllable electrodes, according to another embodiment of the invention;

FIG. 3A is a block diagram schematically representing an electrosurgical system including a control unit in communication with a handpiece having a plurality of electrodes and a plurality of temperature sensors, according to an embodiment of the invention;

FIG. 3B is a block diagram schematically representing an electrosurgical system including a vacuum unit in communication with a handpiece, according to another aspect of the invention;

FIG. 4 is a block diagram schematically representing a handpiece having a cooling unit and a vibration unit, according to another embodiment of the invention;

FIGS. 5A-B each show a plan view of a handpiece, as seen from above, according to two different embodiments of the invention;

FIG. 5C is a side view of the handpiece of FIG. 5A or 5B;

FIG. 5D is a sectional view of the handpiece of FIGS. 5A-C, as seen along the line 5D-5D of FIG. 5C;

FIG. 5E shows a plan view of the underside of the handpiece of FIG. 5A, as seen along the line 5E/F-5E/F of FIG. 5C;

FIG. 5F shows a plan view of the underside of the handpiece of FIG. 5B, as seen along the line 5E/F-5E/F of FIG. 5C;

FIG. 6 is a sectional view of a handpiece, according to another embodiment of the invention;

FIG. 7 schematically represents a spiral of electrically conductive material for forming an electrode, as seen in plan view, according to another embodiment of the invention;

FIG. 8A schematically represents a spiral inductor, as seen in plan view, according to another embodiment of the invention;

FIG. 8B schematically represents a spiral inductor, as seen in plan view, according to another embodiment of the invention;

FIG. 9A schematically represents a portion of a spiral inductor for an electrode, as seen in side view, according to an embodiment of the invention;

FIG. 9B schematically represents a portion of a spiral inductor for an electrode, as seen in side view, according to another embodiment of the invention;

FIG. 10A schematically represents a handpiece, as seen from the side, showing an empty treatment chamber of the handpiece in relation to a target region of skin of a patient, according to one aspect of the invention;

FIG. 10B schematically represents the handpiece of FIG. 10A showing a target tissue of the patient disposed within the treatment chamber, according to another aspect of the invention;

FIG. 11A is a flow chart schematically representing steps in a method for treating a patient, according to another embodiment of the invention; and

FIG. 11B is a flow chart schematically representing steps in a method for selectively heating a target tissue of a patient, according to another embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides methods and apparatus for treating or selectively heating a target tissue of a patient in a non-invasive procedure. As a non-limiting example, the instant invention may be used to selectively heat, remove, and or sculpt adipose tissue, such as subcutaneous fat and/or cellulite. Apparatus of the invention may include a handpiece having a plurality of electrodes, wherein each of the electrodes may be dynamically controlled during a procedure with respect to electrode mode (e.g., active or return), condition (ON or OFF), and power level.

In contrast to prior art devices, apparatus and systems of the instant invention may include a handpiece having a distal flange configured for sealing engagement against an external skin surface, wherein a plurality of electrodes are recessed within a treatment chamber at a location proximal to the flange. Each electrode may be substantially planar, and each electrode may be affixed to and aligned with a treatment surface within the treatment chamber. The configuration of handpieces of the invention may prevent electrode contact with a patient's body or tissue unless a target tissue is drawn into the treatment chamber. The apparatus can be operated in the bipolar and monopolar configurations, or in a combined monopolar-bipolar configuration, and the apparatus can be switched between bipolar configuration and monopolar configuration during a procedure.

In a bipolar configuration, each of the electrodes may be configured to act as an active electrode, or to act as a return electrode, or to be excluded (disconnected) from the system. Each of the electrodes may be dynamically controlled. By dynamically configuring each electrode, e.g., with respect to mode (active or return), condition (i.e., ON or OFF), and power level, various RF field distributions within the tissue are obtained, and thus heating patterns within the target tissue are controllable.

In an embodiment, and in further contrast to the prior art, in the present invention electrodes disposed on or in a handpiece may comprise a spiral inductor, which may be formed from a substantially planar spiral of electrically conductive material. Thus electrodes of the invention may further possess the inherent advantage of evenly distributing electric current density to promote the even heating of treated tissue.

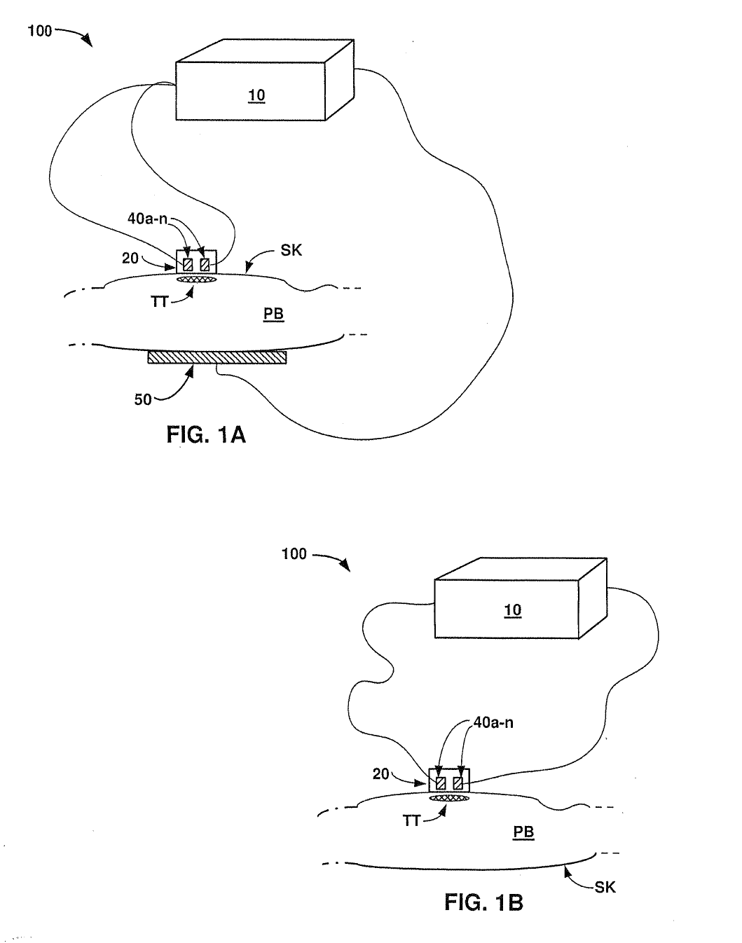

FIG. 1A schematically represents an electrosurgical procedure for treating a patient, according to an embodiment of the invention. Such a procedure may be performed with an electrosurgical system 100, which may include a handpiece 40 in electrical communication with an electrosurgical generator 10. In an embodiment, system 100 may further include a ground pad 50 also in electrical communication with electrosurgical generator 10. In embodiments where ground pad 50 is included in (e.g., connected to) system 100, the procedure may be performed using a monopolar or quasi-monopolar configuration, as described hereinbelow.

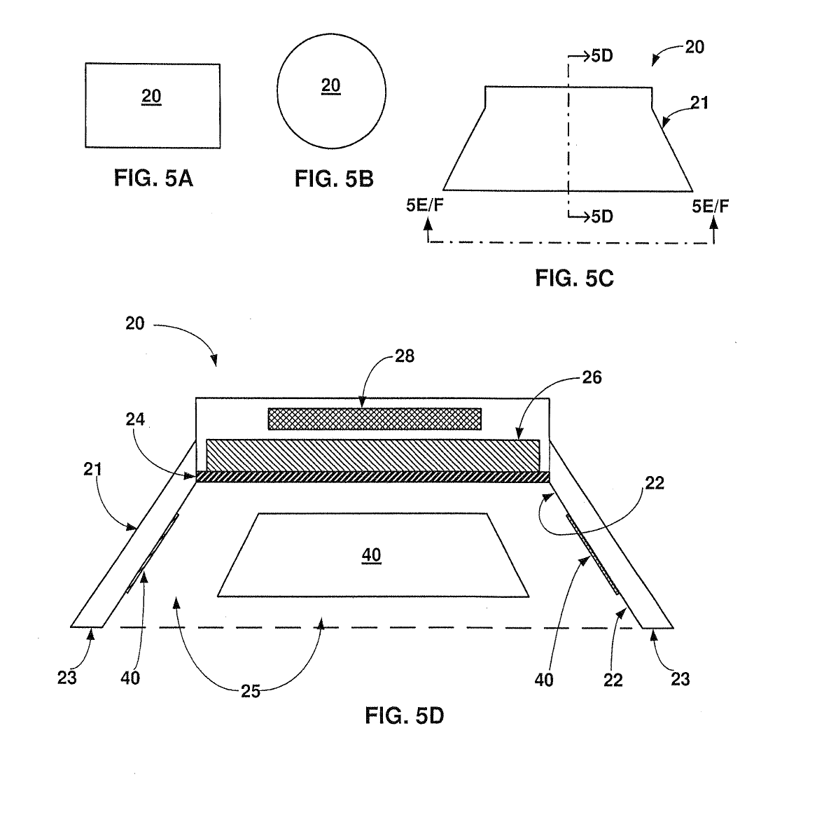

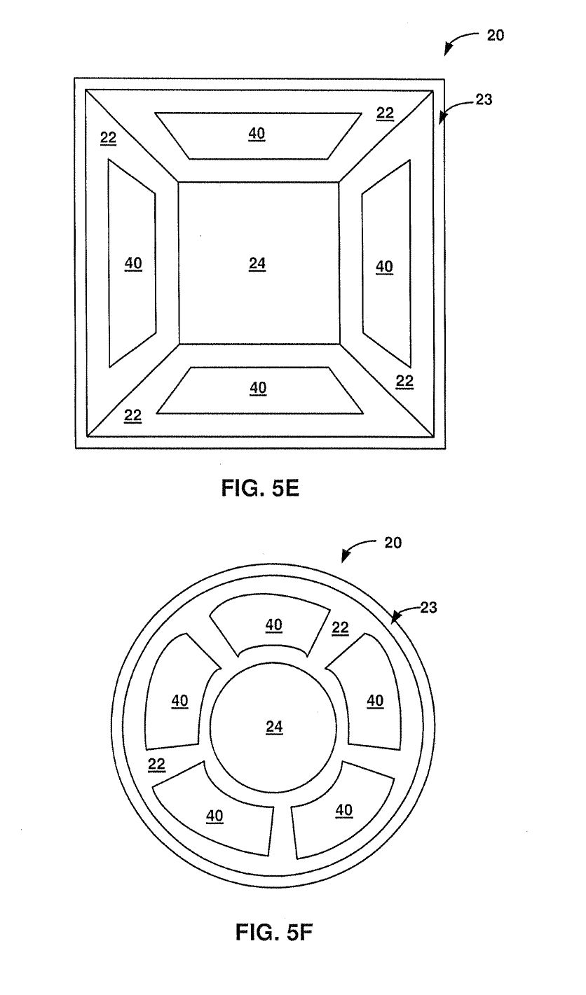

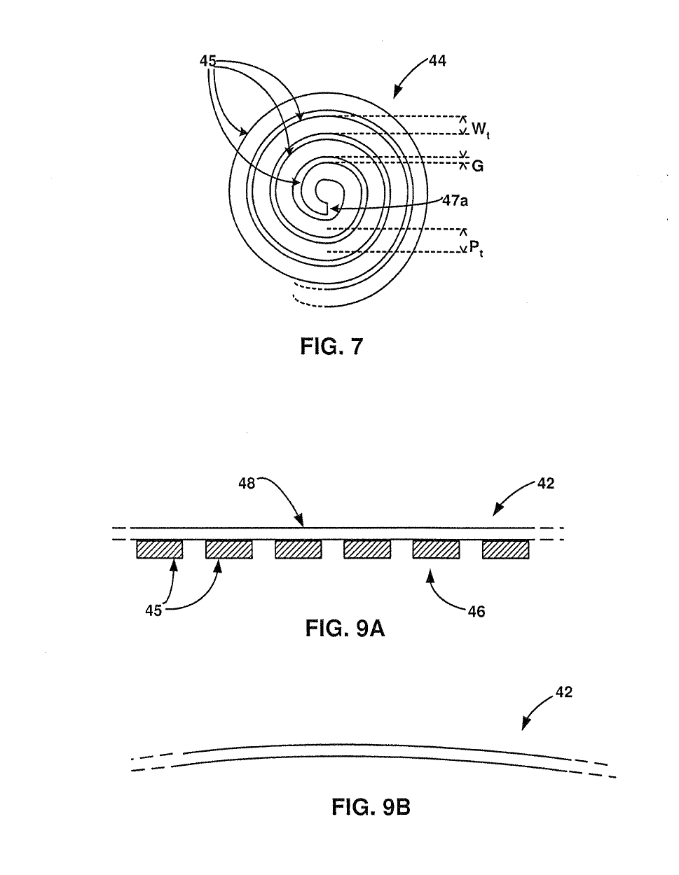

Handpiece 20 may include a plurality of electrodes 40a-n. Although only two of electrodes 40a-n are shown in FIG. 1A, handpiece 20 may include a larger number of electrodes. At least two of electrodes 40a-n may be disposed in at least two different planes within handpiece 20 (see, e.g., FIGS. 5D-F). At least one of electrodes 40a-n may comprise a spiral inductor (see, e.g., FIGS. 7, 8A-9B). In an embodiment, each of electrodes 40a-n may comprise a spiral inductor.

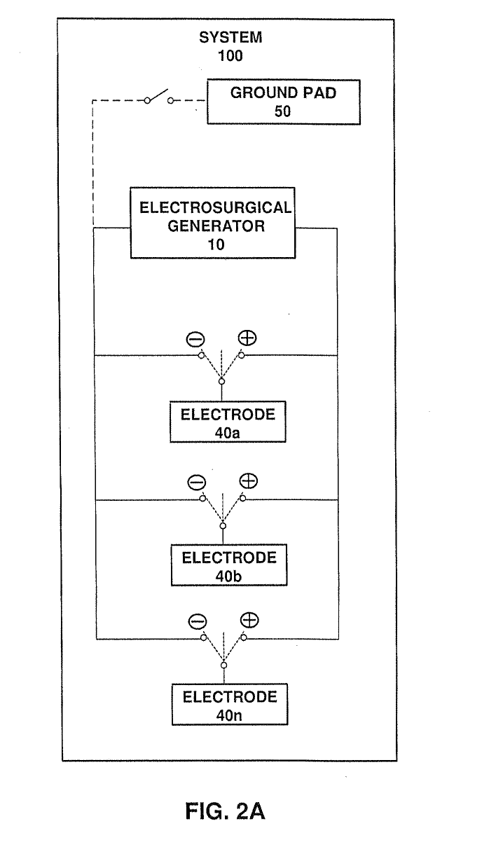

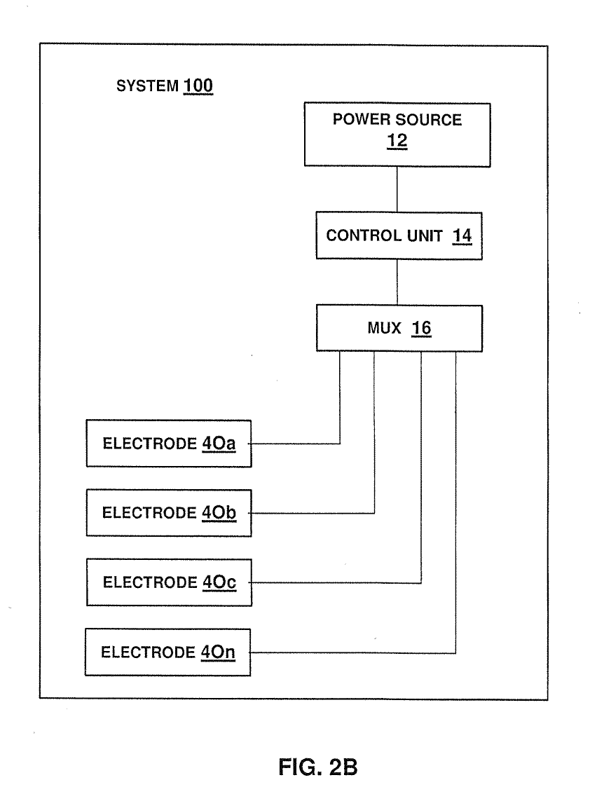

With further reference to FIG. 1A, in an embodiment each of electrodes 40a-n may be configured as an active electrode. In another embodiment, at least one of electrodes 40a-n may be configured as a return electrode. As an example, during a procedure, one or more of electrodes 40a-n may be switched, “on the fly,” between active electrode mode and return electrode mode (see, e.g., FIG. 2A). Further, during a procedure, ground pad 50 may be disconnected from, or re-connected to, electrosurgical generator 10 (see, e.g., FIG. 1B). Such switching of electrode 40a-n between active electrode mode and return electrode mode, as well as connection/disconnection of ground pad 50 to electrosurgical generator 10, may be dynamically controlled according to the nature of the procedure, the electrosurgical parameters, the desired or observed effect(s) on the target tissue, and the like. Accordingly, a given procedure according to the invention may be performed at various stages in a monopolar configuration, a bipolar configuration, or a combined monopolar/bipolar configuration.

Furthermore, each of electrodes 40a-n may be further dynamically controlled, during a procedure, with respect to either an ON condition or an OFF condition. Still further, power or voltage may be dynamically controlled for those electrodes, of electrodes 40a-n, that are configured in the active mode and the ON condition. That is to say, each of electrodes 40a-n may be independently dynamically controlled with respect to active or return mode, ON or OFF condition, and power level.

Handpiece 20 may be adapted or configured for contacting a patient's body, PB. During a procedure, e.g., for removal of excess subcutaneous fat or body sculpting, and the like, handpiece 20 may be placed on the surface of the skin, SK, adjacent to a target tissue, TT. Ground pad 50 may optionally (e.g., according to a desired system configuration) be placed at a location at least somewhat remote from the target tissue. Ground pad 50 may also be referred to as a remote or dispersive return electrode.

In an embodiment, ground pad 50 may comprise a spiral inductor (see, e.g., FIGS. 7, 8A-9B). A return electrode or ground pad comprising a spiral inductor was disclosed in commonly assigned, co-pending U.S. patent application Ser. No. 11/966,895, entitled “High Conductivity Inductively Equalized Electrodes and Methods,” (Atty. Docket No. ALTU-3000), the disclosure of which is incorporated by reference herein in its entirety.

FIG. 1B schematically represents an electrosurgical procedure for treating a patient, according to an embodiment of the invention. Such a procedure may be performed with an electrosurgical system 100, which may include a handpiece 40 in electrical communication with an electrosurgical generator 10. Handpiece 20 may include a plurality of electrodes 40a-n, substantially as described with reference to FIG. 1A. In the embodiment of FIG. 1B, at least a first electrode of electrodes 40a-n may be electrically coupled to a first pole of generator 10, while at least a second electrode of electrodes 40a-n may be electrically coupled to an opposite pole of generator 10. In an embodiment, a ground pad 50 may optionally be brought (e.g., switched) into electrical communication with electrosurgical generator 10 (see, e.g., FIG. 1A). At the same time, each of electrodes 40a-40n may be switched, during a procedure, between active electrode mode and return electrode mode. Accordingly, a given procedure may be performed at various stages in a monopolar configuration, a bipolar configuration, or a combined monopolar/bipolar configuration, while each of electrodes 40a-n may be independently controlled with respect to active or return mode, ON or OFF condition, and power or voltage levels, substantially as described with reference to FIG. 1A.

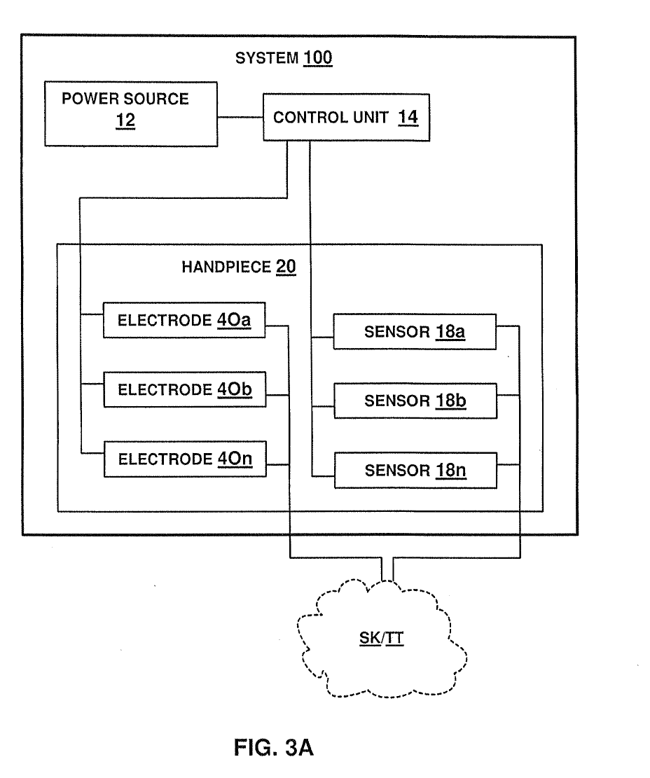

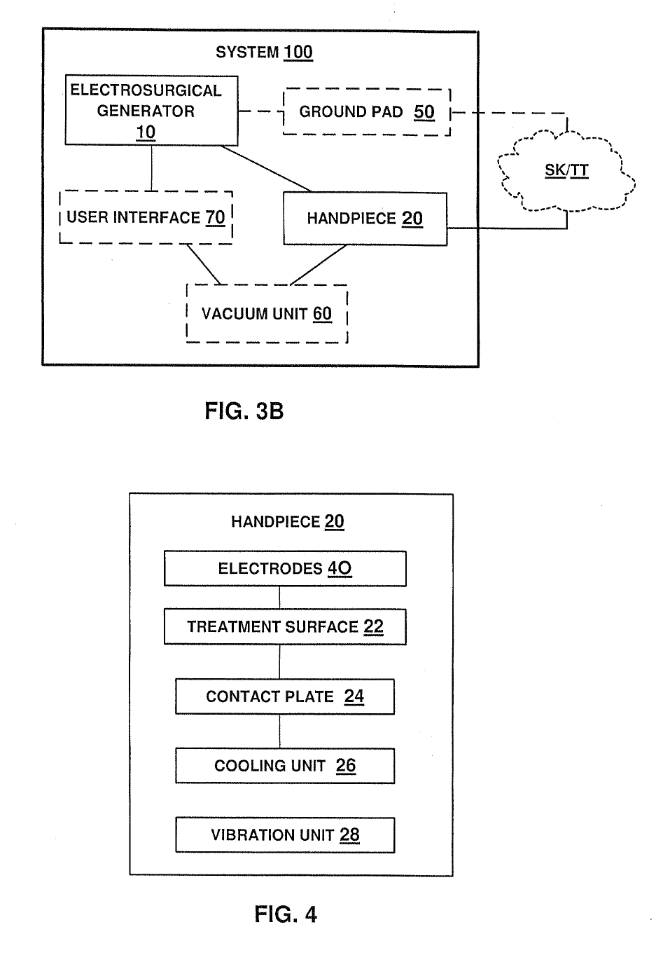

FIG. 2A schematically represents an electrosurgical system, according to an embodiment of the invention. System 100 may include an electrosurgical generator 10 and a plurality of electrodes 40a-n. Each of electrodes 40a-n may be independently controllable with respect to an active electrode mode or a return electrode mode; and each of electrodes 40a-n may be independently controllable with respect to an ON condition or an OFF condition. For example, each of electrodes 40a-n may be independently switchable between opposite poles (±) of electrosurgical generator 10 (active mode versus return mode), or each of electrodes 40a-n may be independently disconnected from electrosurgical generator 10 (ON/OFF condition). Each of electrodes 40a-n may be further independently controllable with respect to a power level or a voltage level. Such control of electrodes 40a-n, with respect to active or return mode, ON or OFF condition, and power or voltage level, may be achieved via a control unit 14 (see, e.g., FIGS. 2B-3A).

In FIG. 2A, switching of electrodes 40a-n with respect to electrosurgical generator 10 is schematically represented; in practice, such switching may be performed via electronic switches, such as transistors, PIN diodes, and the like. Such electronic switches may be integral with other components, for example, control unit 14.

FIG. 2B schematically represents an electrosurgical system, according to another embodiment of the invention. System 100 may include a power source 12, a control unit 14, a multiplexer 16, and a plurality of electrodes 40a-n. Power source 12 may be configured for providing high frequency (e.g., RF) electrical power to one or more of electrodes 40a-n via control unit 14 and multiplexer 16. Control unit 14 may be configured for independently controlling each of electrodes 40a-n with respect to active or return mode, ON or OFF condition, and power or voltage levels, essentially as described hereinabove. In an embodiment, control unit 14, as well as power source 12, may be integral with electrosurgical generator 10 (see, e.g., FIGS. 1A-2A, 3B). It is to be understood that the invention is not limited to the use of a multiplexer, and that other arrangements and configurations for “switching” electrodes 40a-n are also within the scope of the invention. Further, although FIG. 2B shows four electrodes 40a-n, other numbers of electrodes are also contemplated under the invention.

FIG. 3A is a block diagram schematically representing an electrosurgical system, according to an embodiment of the invention. System 100 may include a handpiece 20, a power source 12, and a control unit 14. Handpiece 20 may include a plurality of electrodes 40a-n. Each of electrodes 40a-n may be configured for contacting the skin of a patient. For example, handpiece 20 may be disposed in relation to a patient's body such that each of electrodes 40a-n contacts a region of skin, SK, adjacent to a target tissue, TT.

Handpiece 20 may further include a plurality of temperature sensors 18a-n. Each of temperature sensors 18a-n may be configured for contacting at least one of the skin and a target tissue of the patient. In an embodiment, temperature sensors 18a-n may be disposed within a treatment chamber (not shown in FIG. 3A) of handpiece 20. Each of temperature sensors 18a-n may be configured for sensing temperature values of a portion of a target region of skin, wherein the target region of skin may be disposed adjacent to the target tissue (e.g., adipose tissue disposed beneath the target region of skin). Each of temperature sensors 18a-n may be in communication with control unit 14 for providing thereto sensed temperature values. Each of electrodes 40a-n may be in communication with control unit 14, and control unit 14 may be in communication with power source 12. (In an alternative configuration (not shown), electrodes 40a-n may be directly coupled to power source 12; the invention is not limited to any particular configuration as shown or described.) Control unit 14 may be configured for independently controlling each of electrodes 40a-n with respect to at least one of mode, condition, and power or voltage. Such control of electrodes 40a-n may be performed dynamically, during a surgical procedure, e.g., in response to temperature values sensed by at least one of temperature sensors 18a-n.

In an embodiment, handpiece 20 may further include a cooling unit 26 (see, e.g., FIGS. 4 and 5D). Cooling unit 26 may be in communication with control unit 14, and control unit 14 may be configured for regulating cooling unit 26, for example, responsive to temperature values sensed by temperature sensors 18a-n.

Although FIG. 3A shows three temperature sensors 18a-n, other numbers of sensors are also within the scope of the invention. Similarly, although FIG. 3A shows three electrodes 40a-n, other numbers of electrodes are also within the scope of the invention. As a non-limiting example, handpiece 20 may include from two to ten or more electrodes 40a-n, usually from two to eight electrodes 40a-n, and often from two to six electrodes 40a-n. At a given time during a procedure, at least one of electrodes 40a-n may be in the ON condition. Similarly, at a given time during a procedure, at least one of electrodes 40a-n may be in the active electrode mode. Optionally, at a given time during a procedure, at least one of electrodes 40a-n may be in the return electrode mode.

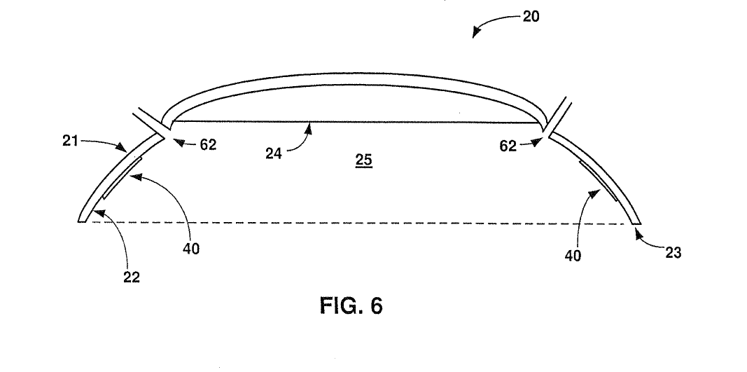

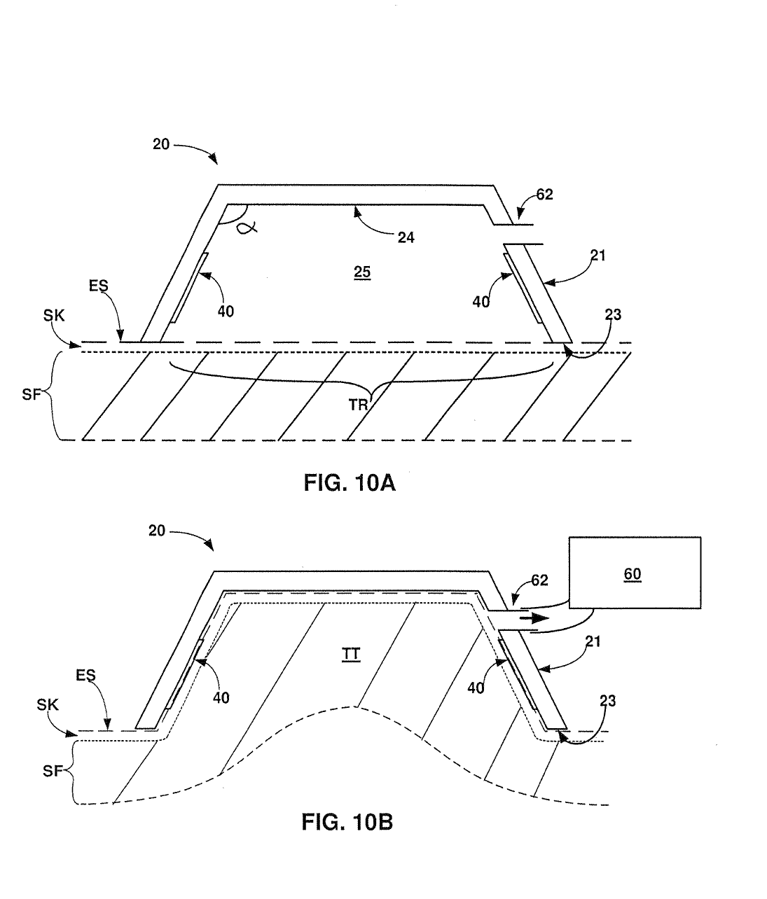

FIG. 3B is a block diagram schematically representing an electrosurgical system, according to an embodiment of the invention. System 100 may include a handpiece 20 coupled to an electrosurgical generator 10. Handpiece 20 may be configured for contacting the patient's body, PB, such as a region of the skin, SK, located above or adjacent to a target tissue, TT, of the patient. Handpiece 20 may include various elements and characteristics as described hereinabove (e.g., with reference to FIG. 3A) and as described hereinbelow (e.g., with reference to FIGS. 5A-6). In an embodiment, system 100 may further optionally include a vacuum unit 60. Vacuum unit 60 may be in fluid communication with handpiece 20, and handpiece 20 may be configured for drawing a target tissue within a void or treatment chamber of handpiece 20 (see, e.g., FIGS. 6 and 10A-B).

With further reference to FIG. 3B, in an embodiment system 100 may further optionally include a remote return electrode or ground pad 50, which may be configured for contacting the skin of the patient. System 100 may optionally be switched between a monopolar configuration and a bipolar configuration during at least a portion of a procedure. For example, ground pad 50, if any, may be switched in or out of system 100 according to the treatment parameters, the type of procedure, sensed tissue temperature values, and the like, substantially as described hereinabove.

With still further reference to FIG. 3B, in an embodiment system 100 may further include a user interface 70. User interface 70 may be coupled to, or in signal communication with electrosurgical generator 10, for inputting power or voltage levels, or other electrical parameters, to electrosurgical generator 10. User interface 70 may also be coupled to, or in signal communication with, vacuum unit 60, for qualitatively and/or quantitatively controlling the application of suction, via vacuum unit 60, to handpiece 20 (see, e.g., FIG. 10B).

FIG. 4 is a block diagram schematically representing a handpiece, according to another embodiment of the invention. Handpiece 20 may include a plurality of electrodes 40a-n, a treatment surface 22, and a contact plate 24. Electrodes 40a-n may be disposed on treatment surface 22. Contact plate 24 may be at least substantially planar. Contact plate 24 may be contiguous with treatment surface 22. In an embodiment, handpiece 20 may further include a cooling unit 26. Cooling unit 26 may be disposed against or adjacent to contact plate 24. In an embodiment, cooling unit 26 may be disposed at least substantially parallel to contact plate 24. In an embodiment, handpiece 20 may still further include a vibration unit 28 configured for vibrating handpiece 20 during a procedure.

FIG. 5A is a plan view of a handpiece, as seen from above, according to an embodiment of the invention. In the embodiment of FIG. 5A, handpiece 20 may have a substantially square or rectangular shape or outline. FIG. 5B is a plan view of a handpiece, as seen from above, according to another embodiment of the invention. In the embodiment of FIG. 5B, handpiece 20 may have a substantially circular or round shape or outline. Of course, other shapes or outlines for handpiece 20 are also within the scope of the invention.

FIG. 5C is a side view of the handpiece of FIG. 5A or 5B. Handpiece 20 may include a shell 21. Further detail of handpiece 20 is shown in FIGS. 5D-F (infra).

FIG. 5D is an enlarged sectional view of the handpiece of FIGS. 5A-C, as seen along the line 5D-5D of FIG. 5C. Handpiece 20 may include a shell 21, a flange 23, a treatment surface 22, and a contact plate 24. Handpiece 20 may further include a plurality of electrodes 40a-n. Each of electrodes 40a-n may be affixed to and aligned with at least a portion of treatment surface 22. Flange 23 may define a distal rim of handpiece 20, and electrodes 40 may be disposed proximal to flange 23. For example, electrodes 40 may be recessed within shell 21/treatment chamber 25 such that electrodes 40 lack contact with the patient's tissue/body when flange 23 is disposed on the patient's skin (see, e.g., FIG. 10A).

In an embodiment, treatment surface 22 may comprise an electrically insulating or dielectric material. In an embodiment, handpiece 20 may further include a plurality of temperature sensors 18a-n (see, e.g., FIG. 3A). One or more portions of treatment surface 22, and at least one of electrodes 40a-n, may be at least substantially planar. Treatment surface 22 and contact plate 24 may jointly define a treatment chamber 25. Handpiece 20 may be configured for receiving a portion of a target tissue within treatment chamber 25 during a procedure (see, e.g., FIG. 10B).

In an embodiment, handpiece 20 may further include a cooling unit 26. Cooling unit 26 may be configured for cooling contact plate 24. Contact plate 24 may be at least substantially planar. Contact plate 24 may be contiguous with treatment surface 22. Cooling unit 26 may be disposed against or adjacent to contact plate 24. In an embodiment, cooling unit 26 may be disposed at least substantially parallel to contact plate 24. Contact plate 24 may be configured for cooling a portion of the patient's skin during a procedure. In an embodiment, cooling unit 26 may comprise a thermoelectric cooler (not shown). The cold side of such a thermoelectric cooler (TEC) may be disposed against or adjacent to contact plate 24, and the hot side of the TEC may be cooled via fluid (e.g., water) flow (not shown). Cooling unit 24 may be configured for cooling contact plate 24 to a temperature down to zero (0°), typically to a temperature in the range of zero (0°) to about 30C, usually to a temperature in the range of about 10° to 25° C., and often to a temperature in the range of about 16° to 22° C..

In an embodiment, handpiece 20 may still further include a vibration unit 28. As a non-limiting example, vibration unit 28 may comprise an eccentric rotor (not shown). During a procedure, vibration unit 28 may be driven or activated to vibrate at least one of handpiece 20 and target tissue disposed within treatment chamber 25.

FIG. 5E shows a plan view of the handpiece of FIG. 5A, as seen along the line 5E/F-5E/F of FIG. 5C. In the embodiment of FIG. 5E, handpiece 20 may have a substantially square or rectangular shape or outline. FIG. 5E shows flange 23, contact plate 24, and a plurality of electrodes 40 disposed on treatment surface 22. Flange 23, contact plate 24, and treatment surface 22 jointly define treatment chamber 25 (see, e.g., FIGS. 5D and 10A). In the embodiment of FIG. 5E, treatment surface 22 may occupy at least two different planes. At least two of electrodes 40 may be disposed in at least two different planes within treatment chamber 25. In an embodiment, each plane, or each substantially planar portion of treatment surface 22, may have a separate electrode 40 disposed thereon. Electrodes 40 may be configured to accommodate various geometries of treatment surface 22. Each electrode may have a substantially elongate or rectangular shape.

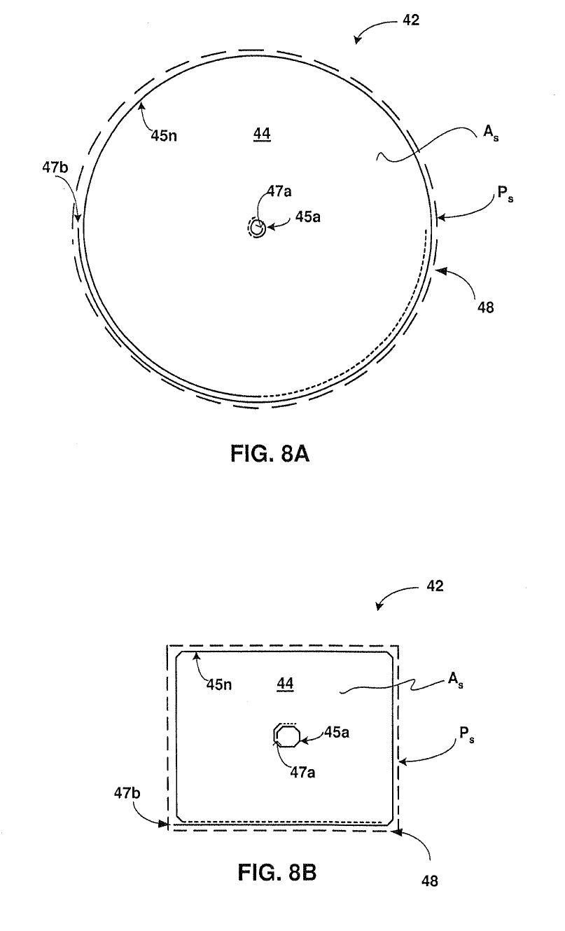

In an embodiment, at least one of electrodes 40 may comprise a spiral inductor 42 (see, e.g., FIGS. 8A-B). In some embodiments, each of electrodes 40 may comprise a spiral inductor. Each spiral inductor may comprise a spiral 44 of an electrically conductive metal (see, e.g., FIGS. 7 and 8A-9B). An active electrode comprising a spiral inductor was disclosed in commonly assigned, co-pending U.S. patent application Ser. No. 11/966,895, entitled “High Conductivity Inductively Equalized Electrodes and Methods,” (Atty. Docket No. ALTU-3000), the disclosure of which is incorporated by reference herein in its entirety.

In an embodiment of the instant invention, each spiral inductor may have a substantially trapezoidal shape, e.g., comprising a quadrilateral outline having two parallel sides and two non-parallel sides. A spiral electrode having such a quadrilateral outline may also have rounded corners (not shown). In the embodiment of FIG. 5E, treatment chamber 25 may have a substantially frusto-pyramidal (truncated pyramid) shape.

FIG. 5F shows a plan view of the handpiece of FIG. 5B, as seen along the line 5E/F-5E/F of FIG. 5C. In the embodiment of FIG. 5F, handpiece 20 may have a substantially round or circular shape or outline. FIG. 5A shows flange 23, contact plate 24, and a plurality of electrodes 40 disposed on treatment surface 22. Flange 23, contact plate 24, and treatment surface 22 may jointly define treatment chamber 25 (see, e.g., FIG. 10B). Each electrode 40 may be configured to accommodate various geometries of treatment surface 22. In an embodiment, one or more of electrodes may comprise a spiral inductor 42, substantially as described with reference to FIG. 5E. As shown, each spiral inductor/electrode 40 may have a substantially arcuate shape or outline. Other shapes and outlines for spiral inductors/electrodes 40 are also within the scope of the invention.

In the embodiment of FIG. 5F, treatment chamber 25 may typically have a substantially frusto-conical (truncated cone) shape. However, it is to be understood that the invention is by no means limited to a handpiece having a treatment chamber of a particular shape or geometry.

In an embodiment, handpiece 20 may include a treatment surface 22 configured for contacting an area of the external surface of the skin of at least about 10 cm², and often treatment surface 22 may be configured for contacting an area of the external surface of the skin of at least about 100 cm². Handpiece 20 may further include various other elements, features and characteristics, e.g., as described with reference to FIGS. 3A, 4, and 6.

FIG. 6 is a sectional view of a handpiece, according to another embodiment of the invention. In the embodiment of FIG. 6, handpiece 20 may include a shell 21, a treatment surface 22 within shell 21, a flange 23, and a contact plate 24. Treatment surface 22 may be contiguous with contact plate 24. Handpiece 20 may further include a plurality of electrodes 40. Electrodes 40 may be disposed on a treatment surface 22.

Treatment surface 22 and contact plate 24 may jointly define a treatment chamber 25 within handpiece 20. Treatment chamber 25 may be configured for receiving at least a portion of a target tissue of a patient. As a non-limiting example, the target tissue may comprise subcutaneous fat. Treatment chamber 25 may be at least substantially dome-shaped. Handpiece 20 of FIG. 6 may further include various other elements, features and characteristics, e.g., as described with reference to other embodiments of the invention (see, e.g., FIGS. 3A, 4).

Handpiece 20 may still further include at least one suction port 62. Suction port 62 may be in fluid communication upstream with treatment chamber 25; suction port 62 may be in fluid communication downstream with a vacuum unit 60 (see, e.g., FIGS. 3B, 10B). Flange 23 may define a lower perimeter of treatment chamber 25. Flange 23 may be adapted for sealing engagement with a region of skin of the patient. Activation of vacuum unit 60 may draw a target tissue within treatment chamber 25, such that the patient's tissue, e.g., skin, makes contact with treatment surface 22 and electrodes 40 (see, e.g., FIG. 10B).

FIG. 7 schematically represents a spiral of electrically conductive material, as seen in plan view, according to another embodiment of the invention. Spiral 44 may include a plurality of turns 45 and an inner terminus 47a. In an embodiment, each electrode 40 of handpiece 20 (see, e.g., FIGS. 5D-F) may comprise a spiral 44. Although spiral 44 of FIG. 7 is shown as substantially round, other configurations are also within the scope of the invention. As an example, spiral 44 may comprise a spiral trace of an electrically conductive metal, such as Cu, Al, or various alloys, as non-limiting examples. In an embodiment, spiral 44 may comprise a filament of the electrically conductive metal, wherein the filament may be disposed on a support layer 48 (see, e.g., FIGS. 8A-9B). Only a few of the radially inner turns of spiral 44 are shown in FIG. 7, whereas spiral 44 in its entirety may comprise from about 10 to 200 or more turns, typically from about 10 to 150 turns, and often from about 15 to 100 turns.

As shown in FIG. 7, spiral 44 may have a pitch, Pₜ, representing a radial distance between the radial midpoints of adjacent turns 45. The pitch of spiral 44 may be in the range of from about 0.1 mm to 10 mm or more, typically from about 0.2 mm to 9 mm, often from about 0.25 to 5 mm, and in some embodiments from about 0.3 to 1.5 mm. In an embodiment, the pitch of spiral 44 may be constant or substantially constant. In other embodiments, the pitch of spiral 44 may vary.

Turns 45 of spiral 44 may have a width, Wₜ, wherein the width, Wₜ is a radial distance across each turn 45. The width of each of turns 45 may typically be in the range of from about 0.05 mm to 10 mm or more, typically from about 0.15 to 9 mm, often from about 0.2 to 5 mm, and in some embodiments from about 0.25 to 1.5 mm. In an embodiment, the width of the various turns 45 may be constant or substantially constant. In other embodiments, the width of turns 45 may vary. A profile or cross-sectional shape of turns 45 may be substantially rectangular or rounded; typically the width of each turn 45 may be greater than its height.

A gap, G may exist between adjacent turns 45 of spiral 44, wherein the gap may represent a radial distance between opposing edges of adjacent turns 45. The gap is typically less than the pitch, usually the gap is substantially less than the pitch, and often the gap is considerably less than the pitch. The gap between turns 45 of spiral 44 may typically be in the range of from about 0.1 mm to 0.5 mm, usually from about 0.15 to 0.4 mm, and often from about 0.15 to 0.3 mm. In an embodiment, the gap between adjacent turns 45 may be constant or substantially constant, even though the pitch may be variable.

FIG. 8A schematically represents a spiral inductor, as seen in plan view, according to another embodiment of the invention. Spiral inductor 42 of FIG. 8A may have a substantially circular or oval configuration. Spiral inductor 42 may include a spiral trace 44 of electrically conductive metal including an inner terminus 47a, and an outer terminus 47b. In an embodiment, spiral inductor 42 may further include a support layer 48, wherein spiral 44 may be disposed on support layer 48 (see, e.g., FIGS. 9A-B). In an embodiment, support layer 48 may comprise an electrically insulating or dielectric material.

Spiral inductor 42 may include a plurality of turns, from a first turn 45a (radially innermost) to an n^{th} turn 45n (radially outermost). In an embodiment, n may be from about 10 to 200 or more, substantially as described hereinabove. Spiral inductor 42 may have a perimeter, Pₛ, and an external surface area Aₛ defined by the perimeter. The electrically conductive metal of spiral 44 may occupy at least about 50% of a total surface area Aₛ, that is to say, at least about 50 percent (%) of the external surface area of spiral inductor 42 may be occupied by spiral 44. Typically, electrically conductive metal of spiral 44 may occupy from about 60 to 99% of external surface area, Aₛ; usually from about 70 to 99% of external surface area, Aₛ; often from about 75 to 98% of external surface area, Aₛ; and in some embodiments electrically conductive metal of spiral 44 may occupy from about 85% to 97% of external surface area, Aₛ.

FIG. 8B schematically represents a spiral inductor, as seen in plan view, according to another embodiment of the invention. Spiral inductor 42 may include a spiral trace 44 of electrically conductive metal having an inner terminus 47a, an outer terminus 47b, and a plurality of turns, 45a-n, substantially as described for the embodiment of FIG. 8A. Spiral inductor 42 of FIG. 8B may have a substantially square or rectangular configuration, a perimeter, Pₛ, and a surface area Aₛ defined by the perimeter. Spiral inductor 42 may include a spiral trace 44 of electrically conductive metal. Spiral trace 44 may occupy a percentage of surface area, Aₛ generally as described with reference to FIG. 8A.

It is to be understood that spiral inductor 42 is not limited to a substantially round or rectangular configuration; instead other shapes for spiral inductor 42 are also contemplated under the invention (see, e.g., FIGS. 5E-F). As a non-limiting example, each spiral inductor may have a substantially trapezoidal shape, e.g., comprising a quadrilateral outline having two parallel sides and two non-parallel sides (see, e.g., FIG. 5E). A spiral electrode having such a quadrilateral outline may also have rounded corners. In another embodiment, each spiral inductor may have a substantially arcuate shape or outline (see, e.g., FIG. 5F).

In an embodiment, spiral inductors 42 of FIGS. 8A-B may comprise a spiral 44 which may be at least substantially planar. In an embodiment, spirals 44 and spiral inductors 42 of FIGS. 8A-B may have a slightly curved or contoured outline (see, e.g., FIG. 9B). In an embodiment, spirals 44 and spiral inductors 42 may be curved or contoured to some extent to accommodate or match a curved or contoured treatment surface 22 (see, e.g., FIG. 6).

FIG. 9A schematically represents a portion of a spiral inductor 42 for an electrode 40, as seen in side view, according to an embodiment of the invention. (In comparison with FIGS. 8A-B, which show spiral 44 disposed on top of support layer 48, FIG. 9A shows spiral inductor 42 as being inverted.) As shown in FIG. 9A, spiral inductor 42 may be at least substantially planar. FIG. 9B schematically represents a portion of a spiral inductor 42 for an electrode 40, as seen in side view, according to another embodiment of the invention. As shown in FIG. 9B, spiral inductor 42 may be at least slightly curved or contoured in outline. Components of spiral inductor 42 in the embodiment of FIG. 9B may be substantially the same as those shown in FIG. 9A and are omitted from FIG. 9B.

With further reference to FIGS. 9A-B, spiral inductor 42 may comprise a spiral 44 of electrically conductive metal. In an embodiment, spiral inductor 42 may further comprise a support layer 48, wherein spiral 44 may be disposed on support layer 48. In an embodiment, support layer 48 may be disposed on treatment surface 22 of handpiece 20. In another embodiment, spiral 44 may be disposed directly on treatment surface 22 (i.e., support layer 48 may be omitted). In an embodiment, spiral 44 may be affixed to treatment surface 22 via a layer of electrically insulating adhesive. Stated differently, in an embodiment, support layer 48 may comprise such a layer of electrically insulating adhesive. Spiral 44 may include an external surface 46. External surface 46 may be a bare metal surface of electrically conductive metal spiral 44.

In an embodiment, spiral inductor 42 may be configured for direct (e.g., bare metal) contact with the patient. For example, in an embodiment a bare metal external surface 46 of spiral 44 may be configured for contacting the patient. In another embodiment, spiral inductor 42 may include a patient-contacting layer (not shown), comprising electrically conductive or low resistivity material, disposed on spiral 44. A spiral inductor having a patient-contacting layer is disclosed in commonly assigned, co-pending U.S. patent application Ser. No. 11/966,895, entitled “High Conductivity Inductively Equalized Electrodes and Methods,” (Atty. Docket No. ALTU-3000), the disclosure of which is incorporated by reference herein in its entirety.

FIG. 10A schematically represents a handpiece, as seen from the side, according to one aspect of the invention. Handpiece 20 may include elements substantially as described hereinabove, including shell 21, treatment surface 22, flange 23, contact plate 24, treatment chamber 25, at least one suction port 62 in fluid communication with treatment chamber 25, and a plurality of electrodes 40 disposed on treatment surface 22. In an embodiment, treatment surface 22 may disposed at, or subtend, an angle, a, with respect to contact plate 24, wherein angle, a is typically in the range of from about 95 to 175°, usually from about 100 to 165°, and often from about 110 to 160°.

In FIG. 10A, handpiece 20 is disposed against a target region, TR, of the patient's skin, SK, such that flange 23 contacts the external surface, ES, of the skin. In FIG. 10A, suction port(s) 62 may be disconnected from vacuum unit 60 (see, e.g., FIG. 10B), and/or vacuum unit 60 may be idle (off). Accordingly, in FIG. 10A treatment chamber 25 may be seen as empty, e.g., a void that does not contain target tissue of the patient.

In FIG. 10B, suction port(s) 62 may be connected to vacuum unit 60 and/or vacuum unit 60 may be activated (on). Flange 23 may be adapted for sealing engagement with the external surface of the skin. For example, flange 23 may be configured for sealing treatment chamber 25 against the skin. Accordingly, in FIG. 10B target tissue, TT, of the patient may be drawn into treatment chamber 25, such that the patient's skin may contact treatment surface 22 including electrodes 40, and electrodes 40 may at least partially surround the target tissue (see, e.g., FIGS. 5E-F). Electrodes 40 may be disposed proximal to flange 23, i.e., electrodes 40 may be recessed within treatment chamber 25 such that electrodes 40 do not contact with the patient's tissue/skin unless the target tissue is drawn into treatment chamber 25.

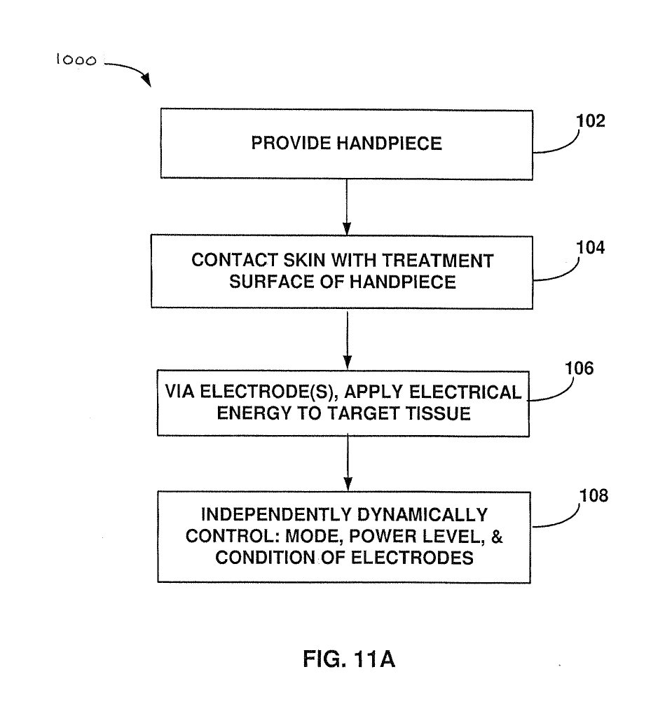

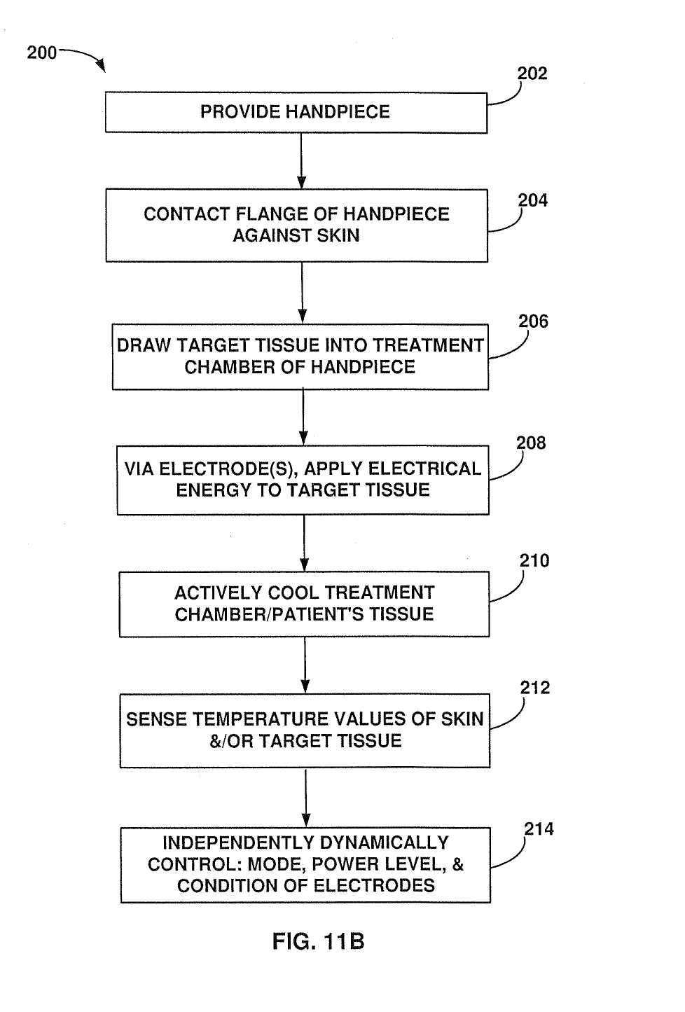

FIG. 11A is a flow chart schematically representing steps in a method 1000 for non-invasively treating a patient, according to another embodiment of the invention. Step 102 may involve providing an electrosurgical handpiece having a plurality of electrodes. The handpiece may include a treatment surface. At least a portion of the treatment surface may be at least substantially planar. In an embodiment, the treatment surface may occupy at least two different planes. One or more of the electrodes may comprise a spiral inductor. Each spiral inductor may be at least substantially planar, and each spiral inductor may be disposed on the treatment surface. In an embodiment, each substantially planar spiral inductor may be disposed on a substantially planar portion of the treatment surface.

Each spiral inductor may be configured for effectively applying electrical energy to the target tissue in a treatment area of the patient. For example, each spiral inductor may be configured for effectively applying electrical energy to subcutaneous fat to provide controlled removal, lipolysis, liquefaction, or atrophy of adipose tissue in the targeted region of the patient's body. Each spiral inductor may comprise at least one spiral of electrically conductive metal, and each spiral inductor may include various other elements, features, and characteristics as described herein, e.g., with respect to FIGS. 7 and 8A-9B.

Step 104 may involve contacting the skin of the patient with the treatment surface. In an embodiment, step 104 may involve contacting the skin against at least one of the electrodes. During step 104, at least one of the electrodes may be brought into at least close proximity to a target issue of the patient. Step 106 may involve applying electrical energy to the target tissue via at least one of the electrodes. As a non-limiting example, the target tissue may comprise adipose tissue, and the electrical energy may be sufficient to controllably remove, ablate, liquefy, or otherwise modify at least a portion of the target tissue.

Step 108 may involve, during step 106, independently dynamically controlling each of the electrodes with respect to at least one of mode of operation, power level, and condition. For example, during step 106 each of a plurality of spiral inductors may be independently dynamically controlled with respect to each electrode being in an active electrode mode or a return electrode mode. At the same time, during step 106 each of the spiral inductors may be independently dynamically controlled with respect to each electrode being in an ON condition (connected) or an OFF condition (disconnected). Similarly, during step 106, each of the spiral inductors may be independently dynamically controlled with respect to a power level of each active electrode in the ON condition.

In an embodiment, method 100 may be used to effectively treat an area of the patient's body of at least about 10 cm², and usually at least about 100 cm². Naturally, in an embodiment the handpiece may be moved in relation to a targeted region of the patient's body during the procedure. Accordingly, the invention may also find applications is treating relatively large regions of a patient's body. The treatment of subcutaneous fat according to method 100 may be cited as a non-limiting example only. Method 100, or modifications thereof, may be applicable to a broad range of different procedures. Any and all variations of method 100, which may be adopted by a skilled artisan in light of applicant's teaching herein, are also within the scope of the invention.

FIG. 11B is a flow chart schematically representing steps in a method 200 for selectively heating a target tissue of a patient, according to another embodiment of the invention. Step 202 may involve providing a handpiece substantially as described with reference to method 100, step 102 (FIG. 11A). The handpiece may include a plurality of electrodes disposed within a treatment chamber of the handpiece. The treatment chamber may be configured for receiving at least a portion of target tissue therein. A treatment surface of the handpiece may define a portion of the treatment chamber. The handpiece may further include a distal rim or flange, which may define a lower perimeter of the treatment chamber. The handpiece may further include various other elements, features, and characteristics as described herein, e.g., with respect to FIGS. 3A, 4, 5D-F and 7.

Step 204 may involve contacting the flange of the handpiece against the external surface of the skin of the patient. The flange may be configured for sealing engagement against the external surface of the skin. In an embodiment, step 204 may involve contacting the patient's skin with the flange such that the flange surrounds a target region of the patient's skin. The target tissue may comprise subcutaneous fat disposed beneath the target region of the patient's skin.

Step 206 may involve drawing the target tissue into the treatment chamber. In an embodiment, the target tissue may be drawn into the treatment chamber via suction applied to the treatment chamber. In an embodiment, step 206 may involve drawing the patient's skin against the treatment surface of the handpiece. Each of the electrodes may be disposed proximal to the distal rim (i.e., flange 23) of the handpiece, wherein the electrodes may be recessed within the treatment chamber such that the patient's tissue/skin does not contact any of the electrodes until the target tissue is drawn into the treatment chamber (see, e.g., FIGS. 10A-B).

Step 208 may involve applying electrical energy to the target tissue via the plurality of electrodes. The electrical energy may be applied to the target tissue during step 206, while the target tissue is disposed within the treatment chamber, such that the target tissue is at least partially surrounded by the electrodes. One or more of the electrodes may comprise a spiral inductor, substantially as described hereinabove (e.g., with reference to method 100, FIG. 11A).

Step 210 may involve actively cooling the treatment chamber of the handpiece, during or after step 208, thereby cooling tissue within the treatment chamber. Such cooling may be accomplished via a cooling unit (see, e.g., FIGS. 4 and 5D). As an example, the cooling unit may be regulated in response to sensed temperature values (step 212, infra) to prevent exposure of patient tissue (e.g., skin) to excessively high temperatures.

Step 212 may involve sensing temperature values for at least one of the target tissue and the patient's skin. Step 214 may involve independently dynamically controlling each of the electrodes with respect to at least one of mode of operation, power level, and condition. Each of the electrodes may be independently dynamically controlled in response to the temperature values sensed at step 212. In an embodiment, step 214 may be performed substantially as described with reference to step 108, method 100 (FIG. 11A). Accordingly, the selective heating of tissue can be dynamically tailored to the treatment of a particular target tissue during any given procedure. In addition, actively cooling (step 210) a non-target tissue during the procedure may also promote the selective heating of target tissue, e.g., subcutaneous fat.

It is to be understood that the foregoing relates to exemplary embodiments of the invention, and that methods and apparatus of the invention may find many applications other than those specifically described herein. Further, none of the examples presented here are to be construed as limiting the present invention in any way; modifications may be made without departing from the spirit and scope of the invention as set forth in the following claims.

What is claimed is:

1. A system for treating a patient, comprising:
a handpiece configured for contacting a target region of skin of the patient, and
an electrosurgical generator coupled to said handpiece, wherein:
said handpiece includes a treatment surface and a plurality of electrodes disposed on said treatment surface, and
said system is configured for independently dynamically controlling at least one of mode of operation, power level, and condition for each of said plurality of electrodes.

2. The system of claim 1, wherein:
at least one of said plurality of electrodes comprises a spiral inductor, and said spiral inductor is at least substantially planar.

3. The system of claim 1, wherein at least two of said plurality of electrodes are disposed in at least two different planes.

4. The system of claim 1, wherein:
said handpiece includes a treatment chamber, and
said handpiece is configured for receiving a target tissue of the patient within said treatment chamber, and said system further comprises:
a control unit, and
a plurality of temperature sensors, wherein:
each of said temperature sensors is in communication with said control unit, each of said temperature sensors is configured for sensing temperature values of a portion of the target region of skin; and
said system is configured for independently dynamically controlling each of said plurality of electrodes in response to said sensed temperature values.

5. The system of claim 1, wherein each of said plurality of electrodes is independently configurable as an active electrode or a return electrode.

6. The system of claim 5, wherein each of said plurality of electrodes is independently switchable between an ON condition and an OFF condition.

7. The system of claim 6, wherein each of said plurality of electrodes is independently dynamically controllable with respect to mode of operation, power level, and condition.

8. The system of claim 6, further comprising:
a ground pad coupled to said electrosurgical generator, wherein:
said system is switchable between a bipolar configuration and a monopolar configuration.

9. The system of claim 8, wherein said ground pad comprises a spiral inductor.

10. The system of claim 1, further comprising:
a treatment chamber disposed within said handpiece; and
at least one suction port in communication with said treatment chamber, wherein each said suction port is disposed in said treatment surface.

11. The system of claim 1, further comprising a vibration unit configured for vibrating at least one of said handpiece and the target tissue.

12. Apparatus for treating a patient, said apparatus comprising:
a handpiece including a plurality of electrodes, wherein:
each of said plurality of electrodes comprises a spiral inductor, and
at least two of said spiral inductors are disposed in at least two different planes.

13. The apparatus of claim 12, wherein:
at least one of said plurality of electrodes is configured for applying electrical energy to a target tissue of the patient, and
each of said plurality of electrodes is configured for independent dynamic control with respect to at least one of mode of operation, condition, and power level.

14. The apparatus of claim 13, wherein:
said mode of operation comprises an active electrode mode or a return electrode mode, and
said condition comprises an ON condition or an OFF condition.

15. The apparatus of claim 12, wherein said handpiece includes:
a contact plate,
a cooling unit configured for cooling said contact plate, and
a treatment surface, wherein:
said treatment surface and said contact plate jointly define a treatment chamber within said handpiece,
said treatment chamber is configured for receiving a target tissue of the patient,
said contact plate is at least substantially planar, and
said contact plate is configured for cooling at least a portion of the target tissue.

16. The apparatus of claim 15, wherein said plurality of electrodes are disposed within said treatment chamber.

17. The apparatus of claim 15, wherein said treatment chamber is at least substantially frusto-conical, frusto-pyramidal, or dome-shaped.

18. The apparatus of claim 15, wherein said treatment surface is disposed at an angle, α, with respect to said contact plate, and wherein said angle is in the range of from about 100° to 165°.

19. The apparatus of claim 18, wherein:
said handpiece includes at least one suction port,
each said suction port is disposed within said treatment surface, and
each said suction port is in fluid communication with said treatment chamber.

20. The apparatus of claim 12, wherein each said spiral inductor includes a spiral comprising an electrically conductive metal.

21. The apparatus of claim 12, wherein each said spiral inductor is at least substantially planar.

22. The apparatus of claim 12, wherein each said spiral inductor has a substantially quadrilateral outline having two parallel sides and two non-parallel sides.

23. The apparatus of claim 12, wherein each said spiral inductor has a substantially arcuate outline.

24. Apparatus for treating a patient, said apparatus comprising:
a handpiece having a treatment surface; and
a plurality of electrodes disposed on said treatment surface, wherein:
said handpiece is configured for contacting said treatment surface against the skin of the patient, and
each of said plurality of electrodes is configured for independent dynamic control with respect to at least one of mode of operation, power level, and condition.

25. The apparatus of claim 24, wherein:
each said electrode is independently configurable as an active electrode or as a return electrode, and
each said electrode is independently switchable between an ON condition and an OFF condition.

26. The apparatus of claim 24, wherein at least a portion of said treatment surface is substantially planar.

27. The apparatus of claim 24, wherein each of said plurality of electrodes is affixed to and aligned with a portion of said treatment surface.

28. The apparatus of claim 24, wherein:
each said electrode comprises a spiral inductor, and
each said spiral inductor is at least substantially planar.

29. The apparatus of claim 24, wherein:
said handpiece includes a flange,
said flange defines a distal rim of said handpiece, and
said electrodes are disposed proximal to said flange.

30. The apparatus of claim 24, wherein:
said handpiece includes a treatment chamber and a flange,
said handpiece is configured for contacting the external surface of the skin of the patient, and
said flange is configured for sealing said treatment chamber against the external surface of the skin.

31. The apparatus of claim 24, wherein said handpiece is configured for contacting said treatment surface against an area of the external surface of the skin, and wherein said area is at least 10 cm².

32. The apparatus of claim 31, wherein said area is at least 100 cm².

33. A handpiece for treating a target tissue of a patient, said handpiece comprising:
a shell having a treatment chamber therein,
a treatment surface within said treatment chamber, and
a plurality of electrodes disposed on said treatment surface, wherein:
said treatment chamber is configured for receiving the target tissue,
said treatment surface is configured for contacting the skin of the patient against said plurality of electrodes, and
each of said plurality of electrodes is configured for independent dynamic control with respect to at least one of mode of operation, power level, and condition.

34. The handpiece of claim 33, wherein each of said plurality of electrodes is at least substantially planar.

35. The handpiece of claim 34, wherein at least one of said plurality of electrodes comprises a spiral inductor.

36. The handpiece of claim 33, wherein said plurality of electrodes are configured for selectively heating one or more regions within the target tissue.

37. The handpiece of claim 33, further comprising:
at least one suction port in communication with said treatment chamber, wherein:
said at least one suction port is configured for drawing the target tissue within said treatment chamber, and
said treatment chamber is substantially frusto-conical, frusto-pyramidal, or dome-shaped.

38. A method for treating a patient, comprising:
a) providing a handpiece having a plurality of electrodes;
b) applying electrical energy to a target tissue of the patient via at least one of said plurality of electrodes; and
c) during step b), independently dynamically controlling each of said plurality of electrodes with respect to at least one of mode of operation, power level, and condition, wherein:
at least one of said electrodes comprises a spiral inductor, and
said spiral inductor is at least substantially planar.

39. The method of claim 38, wherein:
said handpiece includes a treatment surface,
said spiral inductor is disposed on said treatment surface, and the method further comprises:
d) contacting the patient's skin with said treatment surface.

40. The method of claim 3 8, wherein the target tissue comprises subcutaneous fat.

41. The method of claim 38, wherein:
said mode of operation comprises an active electrode mode or a return electrode mode,
said condition comprises an ON condition or an OFF condition, and
said electrical energy is sufficient to controllably remove or otherwise modify at least a portion of the target tissue.

42. A method for selectively heating a target tissue of a patient, comprising:
a) providing a handpiece having a plurality of electrodes, a treatment chamber, and a flange,
b) contacting said flange against the patient's skin, such that said flange surrounds a target region of the patient's skin, and wherein the target tissue comprises subcutaneous fat disposed beneath said target region of the patient's skin;
c) drawing the target tissue into said treatment chamber; and
d) during step c), applying electrical energy to the target tissue via said plurality of electrodes, wherein:
said plurality of electrodes are disposed on a treatment surface within said treatment chamber,
each of said plurality of electrodes comprises a spiral inductor, and
each said spiral inductor is substantially planar.

43. The method of claim 42, further comprising:
e) during step d), sensing temperature values for at least one of the target tissue and the patient's skin; and
f) responsive to said temperature values, independently dynamically controlling each of said plurality of electrodes with respect to at least one of mode of operation, power level, and condition.

44. The method of claim 42, wherein:
said mode of operation comprises an active electrode mode or a return electrode mode, and
said condition comprises an ON condition or an OFF condition.

45. The method of claim 42, further comprising:
g) actively cooling the treatment chamber of the handpiece.

46. The method of claim 42, wherein step c) comprises drawing the target tissue into said treatment chamber such that said plurality of electrodes at least partially surround the target tissue.

47. The method of claim 42, wherein step c) comprises drawing the patient's skin against said treatment surface such that the patient's skin contacts said plurality of electrodes.
